(19) United States
(12) Patent Application Publication
(10) Pub. No.: US 2016/0296241 A1
(43) Pub. Date: Oct. 13, 2016

(54) SURGICAL SAW BLADE

(71) Applicant: Cutting Edge Medical LLC, Louisville, KY (US)

(72) Inventors: Franc Koljaka, Louisville, KY (US); Vasiliy Abramov, Louisville, KY (US); David Phelps, Louisville, KY (US); Dylan Phelps, Anchorage, KY (US)

(73) Assignee: Cutting Edge Medical

(21) Appl. No.: 14/680,407

(22) Filed: Apr. 7, 2015

(51) Int. Cl. A61B 17/16

(52) U.S. Cl. CPC A61B 17/16; A61B 17/1657

(57) ABSTRACT

A surgical oscillating saw blade comprises a drive transmission proximal end, a planer midsection defining an upper and lower surface and a distal cutting edge carrying a plurality of teeth. The teeth are triangularly shaped to define a base, adjacent edges and an apex or tip. The teeth are configured by being bent inwardly or outwardly so that a minimum of tooth and blade surface is in contact with bone during a cutting procedure. The teeth are bent sufficiently to provide a kerf that is greater than the thickness of the blade.

### FIELD OF THE INVENTION

This invention relates to material cutting tools and more particularly to surgical saw blades.

### BACKGROUND OF THE INVENTION

Many orthopedic surgical procedures involve the cutting of bones during arthroplasty or while performing osteotomy and bone re-alignment procedures. In the past, these operations were performed with manual and gigli types of saws. The modern surgical procedures have further evolved by replacing these instruments with electrical powered saws, and especially the oscillating or sagittal saws. These tools impart a back-and-forth oscillating movement to the blade by exerting a cutting action in both directions of the movement. Surgical saws reach cutting speeds of up to 20,000 strokes per minute. Saw blades used in surgical procedures generally have the shape of a rectangular thin sheet metal with cutting teeth in one of the narrow edges and the tool attachment in the opposite end.

Bone cutting presents several problems for the surgeon. The chief problem being an increase in temperature caused by friction during the cutting process. Other problems include excessive vibration and noise which present risks for the surgeon rather than the patient. Therefore, an ideal saw-blade would be one that provides accurate bone cuts without generating too much heat, noise and vibration.

Multiple studies have shown that the rise in temperature of bone during cutting can cause thermonecrosis of the bone resulting in the subsequent failure of the installed implant. The threshold temperature is 47° C. and temperatures above this value may cause osteonecrosis. The time at which bone is held at a temperature value above the threshold is another very important parameter that can affect the response of bone to the cutting operation. It widely accepted in the surgical field that the maximum holding time above 47° C. is 1 minute. Cutting process performed above these levels can result in osteonecrosis with irreversible bone damage and detrimental effects on the solidarity of the implant.

Medical experience and research has shown that the temperature of the bone during cutting without other mitigating actions can increase up to a maximum of 450° C. For this reason, very often surgeons will use saline solution to cool down the cutting area resulting in average temperature of 68C and maximum of about 100° C. These temperatures are still above the threshold of 47° C. and inevitable will negatively impact the bone recovery after the surgery.

Most of the research in avoiding high temperatures during orthopedic surgery has been focused on the drilling tools due to the fact that the drilled holes will host a screw which will support the surgical implant. In this case, osteonecrosis will cause bone failure releasing the screw, and, as consequence, the entire implant may move out of place with serious consequences for the patient. On the other hand, little attention has been paid to the improvement of saw blades since these instruments are generally used in cutting bone fragments. Efforts have been made to develop an internally cooled blade where saline solution is fed between two layers of sheet metal but this invention has not been able to find a practical use in surgical rooms. Most of the development has been focused in modifying the teeth design in order to improve the cutting performance and reduce the heat generation.

The design of the saw blade, especially of the cutting edge, as well as the surface condition of the blade can enhance or reduce the negative factors discussed above. The increase in bone temperature is an effect of the heat generated during the cutting process that, in turn, is the result of the friction forces between the blade and the bone.

The present invention provides a saw blade with a tooth configuration that offers improved cutting action and less thermal buildup, during surgical procedures involving the cutting of bone.

### SUMMARY OF THE INVENTION

The surgical saw blade is generally a planer rectangular body that comprises an improved cutting edge, a midsection and a drive transmission attachment. The drive transmission attachment defines the proximal end of the blade and can be adapted for attachment to a conventional power tool for oscillatory motion. The surface of the drive transmission attachment is not in contact with bone.

The midsection forms the body of the saw blade. The midsection is a generally rectangular planer body defining a pair of longitudinal edges and a distal edge. The midsection will normally not be in contact with bone during the cutting operation and preferably is thinner than the distal edge. The distal edge of the midsection defines the cutting edge of the saw blade.

The cutting edge is configured as a series of triangular blades or teeth having a base disposed along the cutting edge of the blade and adjacent sides culminating at the apex in a tip. The face of each tooth is sharpened at both adjacent sides of the tooth triangle so that the full cutting action of an oscillating or reciprocating blade is achieved. Odd numbered teeth will have sharpened cutting edges on one face of the tooth while even numbered teeth will have cutting edges on the opposite face.

The generation of heat during the cutting process is largely due to friction between bone being cut and the saw blade. In accordance with the invention the teeth are slightly bent inwardly or outwardly. The teeth are bent sufficiently to provide a kerf that is greater than the thickness of the blade body. In this manner there is minimum contact between the bone, the cutting edges of the teeth and the surfaces of the midsection resulting in less heat generated due to friction ring the cutting procedure. In addition debris from the cutting procedure can accumulate between the midsection surface of the blade and the wall of the bone creating more friction and heat buildup. The kerf created by the bent teeth is larger than the thickness of the midsection so that there is little or no contact with the bone and the surface of the midsection. In a preferred embodiment the midsection is thinner than the distal edge of the blade further insuring minimum contact between the bone and the surface of the midsection.

Other advantages and features of the invention will become apparent from following description taken in conjunction with the following drawings.

### BRIEF DESCRIPTION OF THE DRAWINGS

FIG. 1 is a plan view of a typical prior art oscillating surgical saw blade of the type referred to in describing the invention;

FIG. 2A is an enlarged view of a prior art arrangement of teeth for an oscillating surgical saw blade;

FIG. 2B is an end view of FIG. 2A;

FIG. 3A is a sectional side view broken away for purposes of illustration of the cutting edge of a oscillating surgical saw blade showing the arrangement of teeth in accordance with the invention;

FIG. 3B is a sectional side view broken away for purposes of illustration of the cutting edge of an oscillating surgical saw blade showing an alternate arrangement of teeth in accordance with the invention;

FIG. 4A is a sectional side view broken away for purposes of illustration of the cutting edge of the cutting edge of the oscillating surgical saw blade of FIG. 3B during a cutting procedure;

FIG. 4B is a sectional side view broken away for purposes of illustration of the cutting edge of the cutting edge of the oscillating surgical saw blade of FIG. 3B during a cutting procedure; and

FIG. 5 is a side view of a preferred surgical saw blade in accordance with the invention.

### DESCRIPTION OF THE PREFERRED EMBODIMENTS OF THE INVENTION

Certain terminology will be used in the following description for convenience in reference only, and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center of the arrangement. The word “distally” shall mean directed towards the patient, and the word “proximally” shall mean directed away from the patient. Said terminology will include the words specifically mentioned, derivatives thereof, and words of similar import.

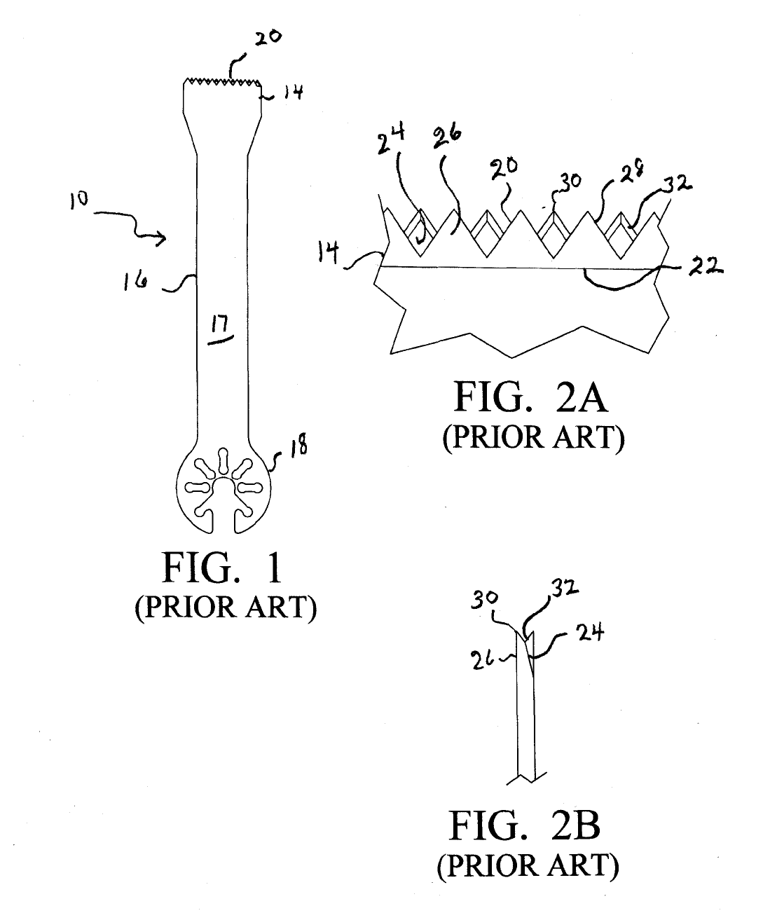

FIG. 1 illustrates a typical oscillating surgical saw blade 10 comprising generally a rectangular body that consists of a cutting edge 14, a midsection 16 defining surfaces 17 and a drive transmission attachment 18. The drive transmission attachment 18 defines the proximal end of the blade and may be of any design for attachment to an oscillatory drive mechanism (not shown).

FIG. 2A is an enlarged view of the cutting end of the saw blade of FIG. 1. A series of aligned triangularly shaped teeth 20 are disposed along the cutting edge 14. Each tooth 20 defines a base 22, a first face 24 and a second face 26 and adjacent edges 28 culminating in the apex of the triangle referred to as the tip 30 of the tooth. The first face 24 and the second face 26 of adjacent teeth 20 are on opposite sides of the tooth. Portions of the first face 24 of each tooth are ground away to the both adjacent edges to form cutting edges 32. As most clearly shown in FIG. 2B grinding the first face 24 results in it being biased in the direction of the second face 26 at the tip 30. As viewed from the blade end after grinding, the tips 30 of adjacent teeth 20 are spaced slightly away from each other and the space there between defines the kerf of the saw blade 10. The distance between the second face 26 of adjacent teeth 20 is essentially equal to the kerf of the saw blade 10. During the cutting operation the second face 26 will contact bone and due to the motion of the saw blade will generate heat caused by friction between the saw blade and the bone being cut.

It has been found that heat generated by the configuration of the teeth of the saw blade of the present invention is unexpectedly less and therefore, there is a lower danger of bone necrosis. In accordance with the invention the improved tooth configuration is achieved by bending the teeth 20 inwardly or outwardly with respect to the base 22 of the tooth.

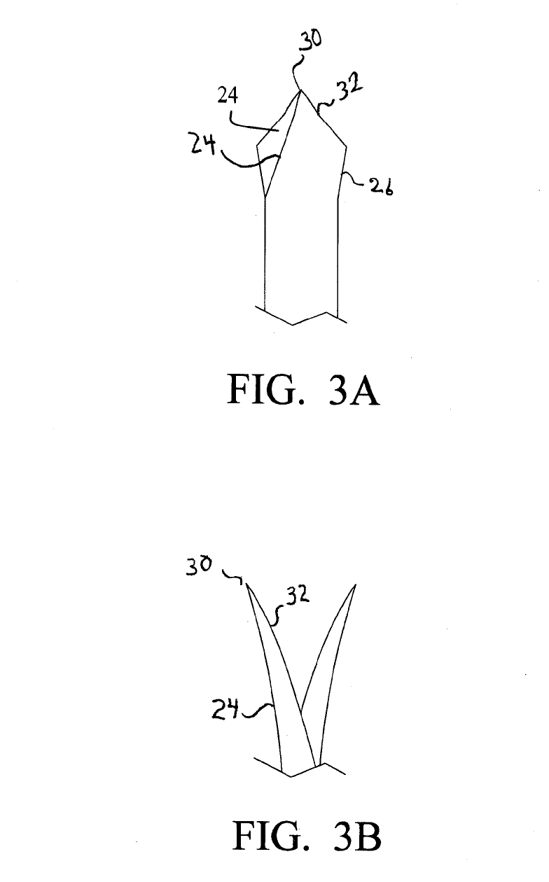

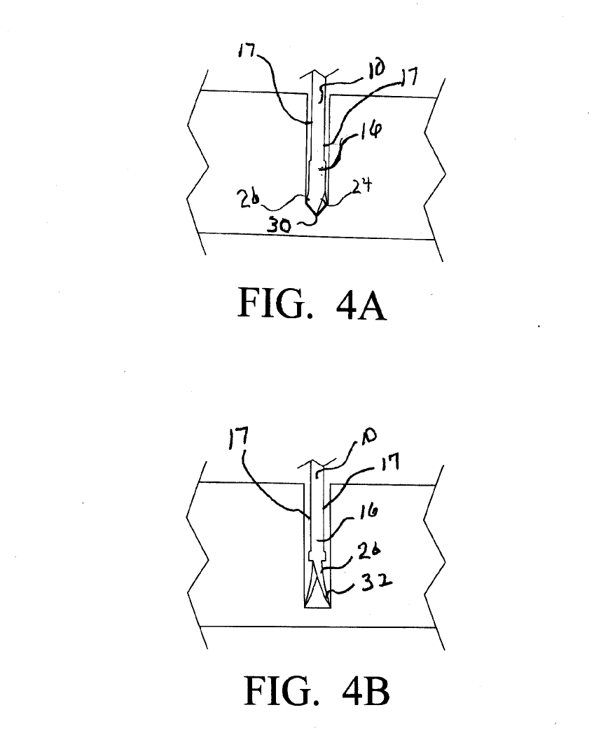

Referring to FIG. 3A the teeth 20 are bent inwardly in the direction of the ground cutting edges 32. The teeth 20 are bent so that the tips 30 of the adjacent teeth are aligned to achieve a single cutting line which allows for a faster cutting speed. As shown in FIG. 4A and as a result of being bent only a small portion of the second face 26 of the teeth 20 are in actual contact with bone. This reduces friction between the second face 26 and the bone and provides space for debris to be pushed out from the cutting edges. In contrast the conventional tooth configuration illustrated in FIG. 2B form two cutting lines and debris will be pushed between the cutting lines. The second faces of the teeth are in abrading contact with the wall of the bone and create frictional heat. The kerf is created by the dimension between the bent second faces 26 of adjacent teeth at the widest point and the kerf is larger than the thickness of the midsection 16 of the blade 10 so the surfaces 17 of the midsection do not abrade bone during a cutting operation.

In an alternate embodiment of the tooth configuration of the invention the teeth are bent outwardly. This configuration is illustrated in FIG. 3B. The tips 30 of the teeth 20 are spaced away from each other a distance of the desired kerf which in any event is larger than the thickness of the midsection 16 of the blade 10. Although there are two cutting lines formed by the spaced apart tips 30 it will be seen from FIG. 4B that only a small portion of the cutting edge 32 is in contact with bone and the major portion of the second face 26 of a tooth 20 will not be in contact with bone.

The midsection 16 defines the body of the blade and is not necessarily involved in the actual cutting of the bone. It and serves merely as a vehicle for the teeth 20. Surfaces 17 may be contiguous with the wall of the bone during the cutting procedure. In the prior art the cutting edge 14 and the midsection 16 are of equal thickness and are substantially equal to the kerf of the surgical saw blade 10. During the cutting procedure the surfaces 17 are substantially contiguous with the wall of the bone and generate heat during the oscillation of the surgical saw blade 10. Moreover additional friction between the surfaces 17 and cut away particles of bone that become lodged between the surfaces and the bone will generate even more heat.

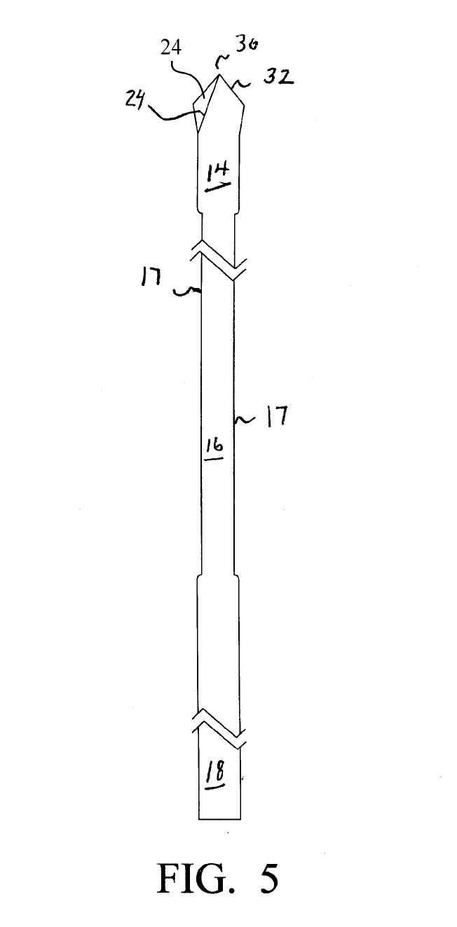

Most conventional surgical saw blades have a midsection thickness equal to the cutting edge. As illustrated in FIG. 5 in a preferred embodiment of the invention the thickness of the midsection 16 is less than the thickness of the cutting edge 14 so that additional space is formed between the surfaces 17 and the bone. In this embodiment the surfaces 17 do not touch the bone and no heat is generated by the surfaces moving against the bone. Additionally the provision of space between the surfaces 17 and the bone allows for better removal of bone chips and reduces yet another source of friction generated heat. As will be apparent from the description that follows, however, it is not critical that the thickness of the midsection 16 be less than the cutting edge 14 as the orientation of the teeth of the saw blade 10 will form a kerf wider than the thickness if the midsection and there will be minimal or no contact between surfaces 17 and bone.

Several factors have to be considered when selecting the material for the surgical blade in view of the medical applications for which the saw blade will be used. Martensitic stainless steel (410 or 420) is preferred for the surgical saw blade. Martensitic stainless steel is preferred because it is resistant to corrosion, can be easily machine in annealed condition, and possesses a high ultimate tensile strength and very good edge-keeping ability in the hardened condition. Surface condition of the surgical blade is a consideration in reducing friction and heat buildup in bone.

Tests have shown that a lower roughness of the metal surface results in lower friction coefficient and, as consequence, lower temperature increase. The best surface condition can be achieved by a polishing process but processing times and associated costs are unjustifiably high. Some products are electro polished but this process, while assuring a low surface roughness, rounds off the cutting edges thus reducing the cutting ability.

Coating the saw blade with a material having a low coefficient of friction, similar to the process commonly used with conventional drill bits, will be beneficial to the goal of maintaining low cutting temperatures. Among coating materials commercially used with drill bits are hard ceramic materials such as titanium nitrate that is applied as a thin layer on the order of 5 micrometers. Titanium nitrate provides a hard low friction surface that reduces heat buildup due to friction. A preferred coating material is an a-C:H material distributed under the trade name Balinit™ Triton. This material provides a thinner, on the order of 0.2 micrometers, hard diamond like, surface having a lower coefficient of friction than titanium nitrate 0.1-0.2 on steel versus 0.4 for TiN. Low friction coatings as applied to the surgical saw blade of the invention further reduces heat buildup due to friction.

From the foregoing it will be seen that the danger of bone necrosis due to frictional heat buildup during a cutting procedure will be reduced by the surgical saw blade of the present invention. The contact between bone and surfaces of the cutting teeth is reduced by bending the cutting teeth to form a configuration in which a large portion the surfaces of the teeth and blade body do not contact the bone during the cutting procedure and thus there is less friction caused by movement of the blade.

1. A surgical saw blade for cutting bone comprising a planar body defining a drive attachment section for attachment of the blade to an oscillatory drive mechanism, a midsection and a cutting edge, said drive attachment defining the proximal end of the saw blade, said midsection extending between said drive attachment and said cutting edge, said cutting edge defining the distal end of the saw blade, said cutting edge comprising a series of aligned triangular teeth having a base, a first face, a second face and adjacent sides biased upwardly away from the base to form a tip, said inner and outer faces being on opposite sides of adjacent teeth and a portion of said first face of each tooth biased to a respective adjacent edge to define a cutting edge, each said tooth being bent so that there is minimum contact between said second face and bone during a cutting operation.

2. The surgical saw blade of claim 1 wherein said teeth are bent inwardly and said tips of said teeth are aligned.

3. The surgical saw blade of claim 2 wherein said second face of said teeth defines the kerf of said surgical saw blade.

4. The surgical saw blade of claim 2 wherein only a portion of said second face contacts bone during the cutting procedure.

5. The surgical saw blade of claim 2 wherein the kerf defined by said second face is greater than the thickness if said midsection of said surgical saw blade.

6. The surgical saw blade of claim 1 wherein said teeth are bent outwardly and said tips of said teeth are spaced apart.

7. The surgical saw blade of claim 6 wherein the kerf of said surgical saw blade is defined by the spacing between the tips of said teeth.

8. A surgical saw blade for cutting bone comprising a planar body comprising a drive attachment section for attachment of the blade to an oscillatory drive mechanism, said drive attachment defining the proximal end of said surgical saw blade, a midsection defining surfaces and a cutting edge defining the distal end of said surgical saw blade, said cutting edge, said cutting edge comprising a series of aligned triangular teeth having a base, a first face, a second face and adjacent sides biased upwardly away from the base to form a tip, said first and second faces being on opposite sides of adjacent teeth and a portion of said first face of each tooth biased to a respective adjacent edge to define a cutting edge, each said tooth being bent to align said tips to form a single cutting line and to bend said second faces of said teeth so that there is minimum contact between said second face and bone during a cutting operation.

9. The surgical saw blade of claim 8 wherein the kerf is defined by the widest point between said bent second faces of adjacent teeth and is greater than the thickness of said midsection.

10. A surgical saw blade for cutting bone comprising a planar body comprising a drive attachment section for attachment of the blade to an oscillatory drive mechanism, said drive attachment defining the proximal end of said surgical saw blade, a midsection defining surfaces and a cutting edge defining the distal end of said surgical saw blade, said cutting edge, said cutting edge comprising a series of aligned triangular teeth having a base, a first face, a second face and adjacent sides biased upwardly away from the base to form a tip, said first and second faces being on opposite sides of adjacent teeth and a portion of said first face of each tooth biased to a respective adjacent edge to define a cutting edge, each said tooth being bent to space said tip away from said tip of an adjacent tooth so that there is minimum contact between said second face and bone during a cutting operation.

11. The surgical saw blade of claim 10 wherein the kerf is defined by the spacing between said tips of adjacent teeth, sad spacing being greater than the thickness of said midsection.
